(19) United States
(12) Patent Application Publication
(10) Pub. No.: US 2026/0051407 A1
(43) Pub. Date: Feb. 19, 2026

(54) METHODS AND SYSTEMS FOR INCENTIVIZING PERSONAL REFLECTION USING A DIGITAL COMMUNICATION NETWORK

(71) Applicant: Sequelae, Inc., Minneapolis, MN (US)

(72) Inventors: Mike Christy, Shakopee, MN (US); Sara Taylor, Edina, MN (US); Scott Archibald, Wayzata, MN (US); Steven J. Catani, Athens, GA (US)

(73) Assignee: Sequelae, Inc., Minneapolis, MN (US)

(21) Appl. No.: 19/282,580

(22) Filed: Jul. 28, 2025

Related U.S. Application Data
(60) Provisional application No. 63/683,052, filed on Aug. 14, 2024.

(51) Int. Cl. G16H 50/30; G06Q 50/00

(52) U.S. Cl. CPC G16H 50/30; G06Q 50/01

(57) ABSTRACT

Methods and systems for incentivizing personal reflection using a digital communication network (DCN) are provided. A DCN is provided for a plurality of members of a population to communicate with each other. An individual member is identified with a lifestyle driven disease and an intervention appropriate for the lifestyle driven disease is facilitated to the individual member. Instructions for the individual member to reflect on using the intervention or lifestyle factors that the intervention allowed the individual member to overcome are provided. A reimbursement is also provided to the individual member for at least some of a cost of the intervention in response to the individual member recording at least one reflection.

### CROSS REFERENCE TO RELATED APPLICATIONS

This application claims the benefit of U.S. Provisional Application No. 63/683,052, filed on Aug. 14, 2024, which application is incorporated herein by reference in its entirety.

### BACKGROUND

Various embodiments relate generally to health care systems, methods, devices and computer programs and, more specifically, relate to incentivize personal reflection using a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

People take actions—whether simple, hard, good, or bad, etc.—that impact their health all the time. Regardless, such actions have consequences that can accumulate over time. However, rarely do people associate such actions with consequences. This may be due to, for example, the lack of data to see the consequence (or the data is available in a way that does not connect to the action). Other times, they may not think about the connection between the action and the consequence. Reflecting on their conditions can help people associate actions with their consequences, thus it is desirable for people to reflect their actions and potential consequences.

Computers have changed the way people interact. Digital networks, which may include the use of social media, allow individuals to interact with others online and connect many people with a community. The online community can be utilized to help build positive behaviors and encourage people to make improvements in their lives. Computers can also provide a way for people to record their actions and review them later as well as to enable a person to reflect on their activities. Reflecting takes conscience effort and is often emotionally draining and cognitively draining. Thus, a person may find it easier to not reflect on such actions.

Thus, what is needed is a way to incentivize user reflection through a digital communication network.

### SUMMARY

Example aspects of the present disclosure include:

A method to manage a health care risk in a population according to at least one embodiment of the present disclosure comprises providing a digital communication network (DCN) for a plurality of members of a population to communicate with each other; identifying an individual member of the population with a lifestyle driven disease; facilitating, through the DCN, an intervention appropriate for the lifestyle driven disease to the individual member; providing, through the DCN, instructions for the individual member to reflect on at least one of using the intervention or lifestyle factors that the intervention allowed the individual member to overcome; and providing a reimbursement to the individual member for at least some of a cost of the intervention in response to the individual member recording at least one reflection.

Any of the aspects herein, wherein the intervention comprises at least one of a medical device, a prescription, and a drug.

Any of the aspects herein, wherein identifying the member comprises at least one of identifying the member based on communications by the member in an online social network of the DCN and receiving an identification of the member from a health care professional team.

Any of the aspects herein, wherein the instructions for the member to reflect comprise instructing the member to reflect using one or more communications in the DCN.

Any of the aspects herein, wherein the one or more communications in the DCN comprise at least one of: one-to-one communications, one-to-many communications, one-to-system communications, system-to-one communications, and system-to-many communications.

Any of the aspects herein, wherein the DCN comprises an artificial intelligence bot configured to derive generated communications from at least one of an analysis of existing communications in the DCN or biometrics provided to the DCN.

Any of the aspects herein, wherein the lifestyle driven disease is at least one of: a metabolic disease, an auto-immune disease, and a cardiovascular disease.

Any of the aspects herein, wherein providing the reimbursement to the member comprises reimbursing the member for at least one of: a total cost of the intervention and out-of-pocket cost of the intervention.

Any of the aspects herein, wherein providing the reimbursement to the member is in response to the member recording at least two reflections.

A method to manage a health care risk in a population according to at least one embodiment of the present disclosure comprises providing a digital communication network (DCN) for a plurality of members of a population to communicate with each other; providing information to the plurality of members of the population about at least one incentive for engaging in a selected activity, wherein the selected activity comprises at least one of: sharing an experience with a self-selected behavior, sharing a reflection on the experience, and sharing an outcome of the experience; analyzing, by the DCN, communications on the DCN to detect an individual member engaging in the selected activity in the DCN; and in response to detecting the individual member engaging in the selected activity, automatically awarding, by the DCN, the at least one incentive into an incentive account of the individual member.

Any of the aspects herein, wherein providing information to the plurality of members comprises sending a communication about the at least one incentive through the DCN.

Any of the aspects herein, wherein the DCN comprises at least one of: an online social network, and a conversation bot.

Any of the aspects herein, wherein detecting the individual member engaging in the selected activity comprises detecting the individual member participating in a behavior.

Any of the aspects herein, wherein the behavior is at least one of a healthy lifestyle behavior, an interaction with a medical system, use of a drug or supplement, use of a medical device, providing a biological sample, and providing a survey or psychological instrument.

Any of the aspects herein, wherein automatically awarding the incentive is in response to detecting the individual member engaging in multiple occurrences of the selected activity.

Any of the aspects herein, further comprising enabling the individual member to use the incentives account to acquire an item that is health inducing.

Any of the aspects herein, wherein the item that is health inducing comprises at least one of: health food, a health monitoring device, a biological test, and an intervention.

Any of the aspects herein, further comprising at least one of determining an appropriateness of the intervention for the member based on communications in the DCN and receiving an indication of an appropriateness of the intervention for the member from a health care professional team.

Any of the aspects herein, wherein the intervention is directed to improving at least one of: a metabolic disease, an auto-immune disease, and a cardiovascular disease.

A method to manage a health care risk in a population according to at least one embodiment of the present disclosure comprises providing a digital communication network (DCN) for a plurality of members of a population to communicate with each other; informing the plurality of members of the population about at least one incentive for engaging in a selected activity, wherein the selected activity comprises participating in a self-selected behavior; analyzing, by the DCN, communications on the DCN to detect an individual member engaging in the selected activity in the DCN; receiving a reflection from the individual member regarding the selected activity; and in response to detecting the individual member engaging in the selected activity and receiving the reflection, automatically awarding, by the DCN, the incentive into an incentive account of the individual member.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The use of “substantially” in the present disclosure, when referring to a measurable quantity (e.g., a diameter or other distance) and used for purposes of comparison, is intended to mean within 5% of the comparative quantity. The terms “substantially similar to,” “substantially the same as,” and “substantially equal to,” as used herein, should be interpreted as if explicitly reciting and encompassing the special case in which the items of comparison are “similar to,” “the same as” and “equal to,” respectively.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as a coupling to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4A shows an example of a computing system, in accordance with one or more embodiments.

FIG. 4B shows an example of a network, in accordance with one or more embodiments.

### DETAILED DESCRIPTION

Medical interventions, such as like pharmaceuticals, can be used over the long-term to mitigate the consequence, severity, or discomfort associated with a disease. In most cases this is the intended use, at least until the underlying disease progresses to the state where an even more powerful intervention may be recommended to a person. This can often be the case even when the disease has a basis in the person's lifestyle as patients and manufacturers may view the pharmaceuticals as a replacement to making lifestyle changes which can be harder to adopt. Finding and adopting lifestyle changes that work is a cognitively and emotionally challenging process that is incompatible for most people while dealing with the underlying disease and its symptom at the same time.

Interventions can be used in ways to allow short-term use of the intervention to give the patient cognitive and emotional room to better appreciate that lifestyle change is possible (and preferred over a lifetime of pharmaceutical use). However, people can seize upon the benefits of the intervention if they take the time to consider their conditions more fully. Various embodiments are directed to incentivize user reflection using a digital communication network (DCN) as reflecting on “what” the use of the intervention helped overcome in a person lifestyle and environment can allow the intervention to be used as a tool rather than a crutch. Enabling a user to earn incentives for lifestyle promoting activities, and then use those incentives to obtain other therapies can help incentivize the user to engage in those activities. Additionally, embodiments may be used to slowing progression of an adverse health condition or to manage a disease.

Various embodiments are directed to inducing reflection in an individual member of a population within a digital communication network (DCN). Reflections are an important part of a person's experience. They allow the person to turn observations about the connection between their actions and the consequences of those actions into useable insight. Digital health applications typically provide people access to data about their health or health-related actions and often allow them to record observations about how they feel. This can cause reflection but in a minimal way.

In some embodiments, the DCN, using a conversation bot, is able to help patients with seeking help for their disease or other health related issue. The conversation bot, using artificial intelligence (AI), can review community discussions on the DCN to identify key issues that may be a concern for an individual member. The bot can ask questions of the individual member so that they can reflect on their situation and/or better appreciate the consequences of any actions. This can help identify conditions, reactions, etc. The conversation bot can also review community discussions to identify possible questions that can, for example, be asked of a doctor to help a patient (e.g., the individual member) with the preliminary work and setting them up to get the key information for proper medical intervention and treatment.

Various embodiments also use AI-driven bots to induce more user reflections. In traditional systems, conversation bots respond to questions with community or expert-sourced answers whereas in embodiments of the present disclosure the conversation bots respond to observations (which may be provided directly or indirectly by the user) with questions designed to cause user reflection on those observations. User reflections may be used by the conversation bot to pinpoint better information for the user, for example, by providing more applicable community or expert-sourced answers. In other embodiments, the individual member reflections may be provided to care givers and/or physicians in order to better evaluate the individual member.

Feedback, interventions, or suggestions to the individual member can be generated and provided by the DCN or other members in the DCN, and such feedback, interventions, or suggestions can be based in part on creating greater awareness of lifestyle changes and medial options. A lifestyle (or behavior) change can be evaluated based on the individual member's reflections. Such induced reflections may be used to help identify the individual member's condition, for example, to evaluate whether a lifestyle change is working.

Additionally, the individual member's reflections can be used to provide the individual member's experience as a social aspect. As a social process, value can be placed on those members of a community that have relevant experience. The reflections from the individual members can be induced in order to help elevate the aspects of the experience that may be more instructional to the community as a whole. This can help increase the number of members of the community thinking about an action, for example, creating an atmosphere where people can learn about others in who have undergone a change they are considering.

The system can also help an individual member in creating posts about their experiences. The AI-driven conversation bots can engage the individual member and propose content for the post based on the individual member's response. The engagement may include inducing reflections and/or analysis of the individual member's health situation. The posts may be for one-to-one communications, such as facilitating the conversation with the conversation bot, or one-to-many communications, such as general posts for the population.

In some embodiments, the conversation bot may also propose content for the post based on other communications in the DCN. By proposing content, the bots can reduce friction for the individual member in creating communications for the DCN. By facilitating such posts, the DCN can increase general awareness in population of health conditions (or risks).

Additionally, the community can be used to help support the individual member in other ways. Lifestyle interventions are inherently safe. They use the philosophy that any action now is preferred to a “better” action later. They also support the concept that ideas and communication are healthcare.

The community can also be used to encourage lifestyle interventions, which are inherently safe. Such community may use the philosophy that any action now is preferred to a “better” action later and also support the concept that ideas and communication are healthcare. Conventionally, lifestyle change has been looked at as in individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical, as will be discussed below.

Conventional lifestyle applications may tell individuals the “right thing to do”, which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual member or members of a population. For example, in a community where a person is a peer, the actions they take and learn from are that their volition and may result in increased agency (or autonomy) or self-efficacy. This not only increase the chances of continuous lifestyle improvement, but improved outcomes throughout the heath system.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working.

Thus, it is desirable to provide systems and methods that incentivize members to use an intervention and to induce reflection regarding the intervention by an individual member whether through the DCN directly or via one or more conversation bots. Such reflections can beneficially provide insight into the individual member's life and lifestyle and whether certain actions or interventions are beneficial to the individual member.

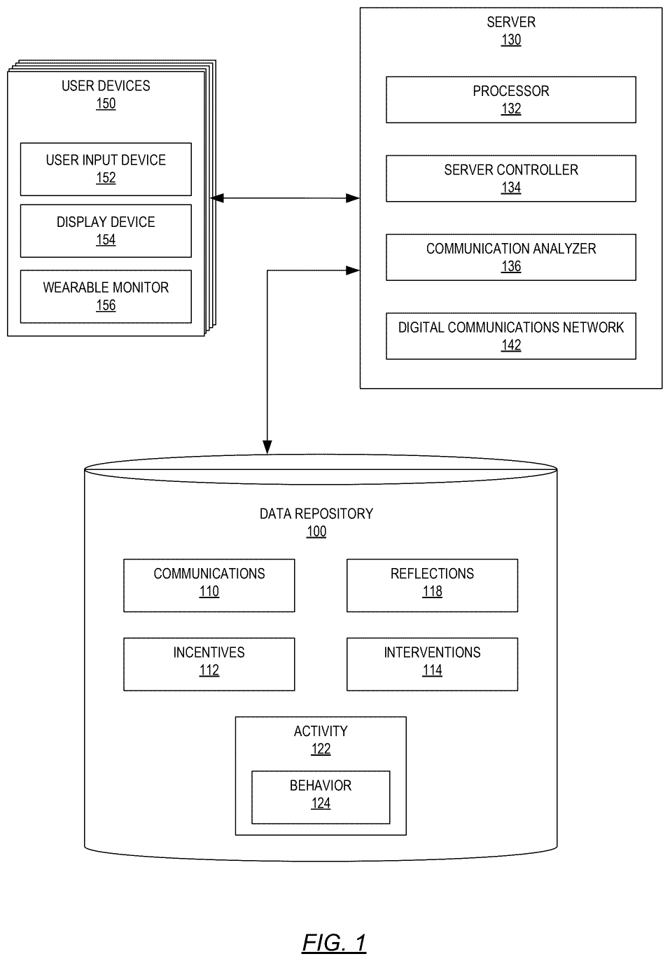

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communication(s) (110). The communications (110) are communications from members in a digital communications network (DCN) (142) or from the DCN (142) (e.g., the DCN as a chatbot). The communications (110) may be, for example, text-based, image-based, multimedia communications, audio communications, or any combinations thereof. The communications (110) may be between members, communications in a forum, communications with the DCN (142), or communications generated by the DCN (142) (e.g., communications from a chatbot). The communications (110) can be one-to-one communications, one-to-many communications, one-to-system communications, system-to-one communications, and system-to-many communications. The communications (110) can include information about a member's experience with a particular intervention (114) and whether the intervention (114) was helpful to the member. The communications (110) can also include one or more reflections (118) from a member such as an individual member. The communications (110) can also include information about an incentive (112) and/or intervention (114), which may be sent to the members of the DCN (142).

The data repository (100) also stores incentive(s) (112). The incentives (112) are discounts, coupons, money, or other rewards to incentivize an identified member to participate in an intervention (114), an activity (122), and/or to provide reflections (118) on their experience with the intervention (114) and/or activity (122). The incentives (112) may be, for example, a discount for the intervention (114) and/or the activity (122). For example, the intervention (114) may be attending a yoga class, and the incentive (112) may be a coupon to cover the cost of the yoga class for the identified user. The incentive (112) may also be an item that is health inducing. The item may be, for example, health food, a health monitoring device, a biological test, and the intervention (114). The incentive (112) may include one or more incentives (112).

The data repository (100) also stores intervention(s) (114). The intervention (114) is an activity, suggestion, or recommendation provided to one or more members with the purpose of improving the one or more member's health. The intervention (114) may also be a health-based activity. Such suggestions or activities may include, for example, lifestyle interventions such as fasting, stress reduction, improving sleep behaviors, improving mood, reducing anxiety, improving relationships, participating in a cardio activity, and/or dietary changes. The interventions (114) may also be a medical intervention such as, for example, prescribing medication or pharmaceuticals to the one or more members. Such medication may be, for example, a drug. The drug may be, for example, a weight loss drug that may be incretin agonist. In still other embodiments, the intervention (114) may be a medical procedure, a medical device, a medical surgery, dietary supplements, or a wearable device or sensor. In some instances, the intervention (114) may be provided to a member's healthcare provider.

The data repository (100) also stores reflection(s) (118). The reflections (118) may be a reflection of the individual member's experience with the intervention (114) and/or activity (122). The reflections (118) may be recorded by the individual member in text, audio, visual media, or any combination thereof. The reflections (118) may also be shared with other members in the DCN (142) via, for example, communications (110).

The data repository (100) also stores activities (122). The activity (122) may be, for example, sharing an experience with a self-selected behavior (124), sharing a reflection on the experience, and/or sharing an outcome of the experience. The behavior (124) may be, for example, a healthy lifestyle behavior, an interaction with a medical system, use of a drug or supplement, use of a medical device, providing a biological sample, and providing a survey or psychological instrument.

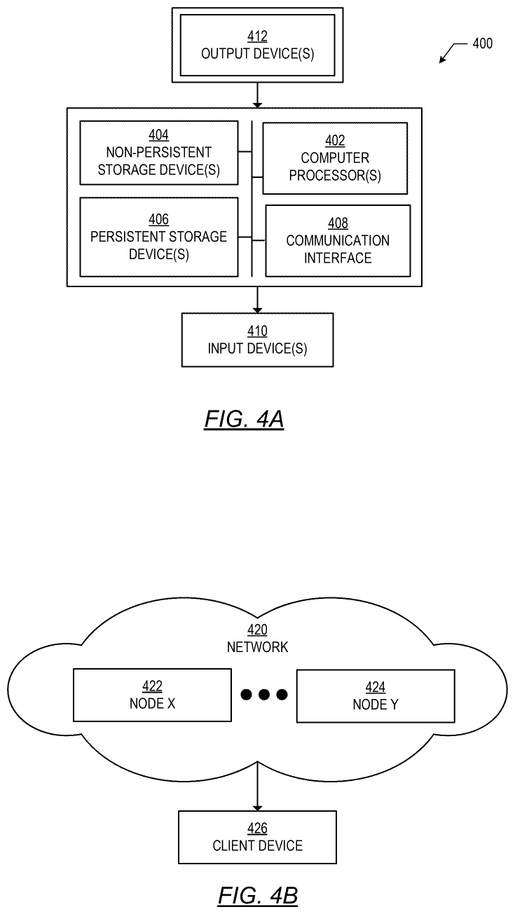

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136). An example of a computer system and network that may form the server (130) is described with respect to FIG. 4A and FIG. 4B.

The server (130) also includes a computer processor (132). The computer processor (132) is one or more hardware or virtual processors which may execute computer readable program code that defines one or more applications, such as the communication analyzer (136). An example of the computer processor (132) is described with respect to the computer processor(s) (402) of FIG. 4A.

The server (130) also may include a server controller (134). The server controller (134) is software or application specific hardware which, when executed by the computer processor (132), controls and coordinates operation of the software or application specific hardware described herein. Thus, the sever controller (138) may control and coordinate execution of the communication analyzer (136).

The server (130) also includes a communication analyzer (136). The communication analyzer (136) is software or application specific hardware which, when executed by the computer processor (132), receives the communications (110) as input and can identify an individual member with a lifestyle driven disease based on the communications (110). The communication analyzer (136) can also generate communications (110) for the DCN (142) to send to members of the population regarding, for example, the intervention(s) (114), incentive(s) (112), activities (122), and/or reflections (118). The communication analyzer (136) can also determine an appropriateness of the intervention (114) for an individual member based on the communications (110).

The server (130) also includes a digital communications network (DCN) (142). The DCN (142) may be, for example, an online social network and/or a conversation bot. The DCN (142) is a network through which members of a population can interact with each other, or with a system supported by the DCN (142). The DCN (142) can also provide means for members of the population to communication with each other. Members can share, for example, their reflections (118) and/or experiences with the intervention (114). The DCN can also be used to deliver or transmit the incentive (112) and/or the intervention (114) to the individual member.

The DCN (142) may be, for example, an artificial intelligence bot configured to derive generated communications from analysis of existing communications (110) in the DCN (142) or biometrics provided to the DCN (142). Biometric data can be used in many instances such as the provisioning and commissioning of healthcare. For example, biometric data can be used to diagnose diabetes, prescribe hypertension medication with a patient's blood pressure exceeds a certain value, prescribe cancer drugs, etc. In another example, cancer drug can be available for reimbursement if a certain genetic signature is present in the tumor. The decision tree and other logic behind these relationships considers many things including what is known about the variance in the intervention's efficacy, therapeutic index, the accuracy and precision in the biometric data's measurement, as well as the cost-benefit ratio of the intervention compared with other interventional options.

The biometric data, as well as reflections from the individual member, can be used in a “feedback” decision tree where, for example, an intervention can be prescribed or recommended to an individual member based on the biometric data and/or the reflections. Then the biometric data or additional reflections can be obtained to study the effects or results of the intervention.

In some embodiments, the DCN (142) uses a conversation bot to help patients with seeking help for medical conditions. The conversation bot is an artificial intelligence (AI) tool that can review community discussions on the DCN (142) to, for example, identify key issues that may be a concern for a patient. The bot can ask questions of the individual member so that they can reflect on their situation and/or better appreciate the consequences of any actions. This can help identify conditions, reactions, etc. The bot can also review community discussions to identify possible questions that should be asked of a doctor to help a patient with the preliminary work and setting them up to get the key information for proper medical intervention and treatment.

In some embodiments, the AI bot may use a visual or graphical interface, for example, a rendered avatar, or a plot or image from the bot or sharing one with the bot. The DCN (142) can send a visual representation showing that person's data from a wearable, their interaction with the community, their consistency in talking with the bot, etc. The AI bot may then ask questions like “What do you notice” or “Is anything here surprising” in order to induce a reflection from the person.

The conversation bot can analyze data from various sources, in part, to formulate its communications with the user. These sources include data from i) the DCN (142), ii) the ongoing conversation with the user, and iii) previous conversations with the user. The conversation bot can also use temporal and situational cues to formulate its communication next with the member. When analyzing the data, the conversation bot may quote sections of the conversations for use with communications between the user and/or the conversation bot may identify patterns in the discussions. When formulating the communications, the conversation bot can analyze exchanges in the DCN (142) from other members. These communications can be limited to specifically target individuals, such as those deemed to be close contacts to the user or members that are considered influencers.

One-to-one communications with the conversation bot can be triggered and informed by one-to-many communications in the DCN (142), and conversations with the conversation bot can (a) refer individuals to the relevant communications in the DCN (142), (b) suggest posts to the DCN (142), and (c) make reference to ideas contained in posts. The two ways to interact can behave in a synergistic way and allow the conversation bot to include a “human in the loop” in a surprisingly scalable way.

During the communication between the user and the conversation bot, the DCN (142) can analyze the user's responses. Based on the input from the user, the DCN (142) may identify an adverse health condition for the user. The conversation bot may then gather more information regarding the adverse health condition, for example, to develop more details to confirm the adverse health condition. The communication may be based on related experiences from another member of the population. The conversation bot may quote content from a post by that other member.

The synergy between the conversation bot and the DCN (142) can facilitate reflection on a topic. Alternatively, this can be seen to be reversed where the act of facilitating reflection synergizes with usage of the bot in the DCN (142).

Using analysis of the user's communications in the DCN (142), the conversation bot can suggest a reflection on a topic in a one-top-one communication. The suggestion may be made as part of a question to the user. The conversation bot may then focus its conversations with that user on that topic for a period of time (e.g., a number of exchanges, a few minutes, a few days, etc.). The conversation bot can incorporate excerpts of communication on the DCN (142) in its responses. The excerpts of communications may include post titles, post topics, quotes from the body of the communication, and/or post contributors. Also, the excerpts could be summaries of communications on the DCN (142). These summaries may be created automatically by the DCN (142) and/or are based on a topic, a member's communications, or a time period.

The bot may prompt the user to reflect on various classes of action. The action may be active or passive. The action may also be one that was contemplated even if not undertaken.

Two or more conversation bots, each with a different personality or a different role (e.g. facilitate reflections, find useful communications, say good morning and inspire a positive start to a day, handle routine network tasks, provide custom communication summaries), may be provided by the DCN (142). The family of bots could each use conversations between the other bots and the user on the DCN (142) to formulate their responses. When working with a user, the bots may be selected based on the personality of the bot that is expected to work best with that user. The decision may be based on past dealings with the user and/or through analysis of that user's communication on the DCN (142). For example, when attempting to induce a reflection, the bot may use a first personality, then switch to a second personality when helping draft a post regarding that reflection. Accordingly, various embodiments can be used to provide a method to improve the communications of the conversation bot through using authentic experiences of the user in the communication.

In some, non-limiting embodiments, the DCN (142) can include use of an VR environment as an intervention, as an alternative channel for conversation bot conversations, and likewise for digital communication posts and conversations. Thus, the use of VR can facilitate reflection. A conversation bot may be used in VR to facilitate reflection on the interventions in the VR environment. As discussed above, the conversation bot may be given a specific personality or role. In VR, the bot may also be provided with an appearance based on the personality or role. Additionally, data from the VR intervention can be used to inform the reflection dialog.

The DCN (142) can analyze communications that are received as text (or audio) which is received via streaming or in batches. The DCN (142) may analyze the communication individually or as a part of a larger conversation. Machine learning (ML) models, or natural language processing (NLP) algorithms may be used to examine the communication (for example, counting frequency of certain words). The DCN (142) produces an output which is used to update the system's understanding of how well someone is doing at reflection.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (400) shown in FIG. 4A) that communicate with the server (130). The user devices (150) may include a wearable monitor (156) and be configured to send stress indicator data (110) to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

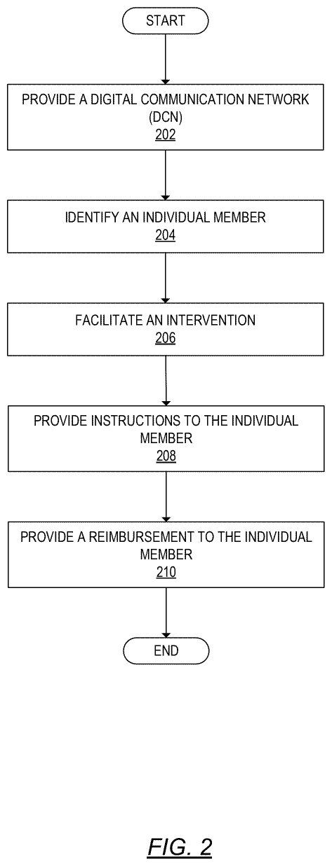

FIG. 2 is a logic flow diagram that illustrates a method, and a result of execution of computer program instructions, in accordance with various embodiments. The method can be used to induce or encourage reflection in an individual member. The method can also, in other instances, be used in part to facilitate or accomplish at least one of: manage the health care cost of the population; reduce the health care risk in the population; and/or slow the progression of an adverse health condition or a lifestyle driven disease. The lifestyle driven disease may be, for example, a metabolic disease, an auto-immune disease, or a cardiovascular disease.

At Block 202, a digital communication network (DCN) is provided. The DCN may be the same as or similar to the DCN (142) and provides a network for members of a population to interact with each other or with the DCN (142).

At Block 204, an individual member is identified. Identifying the individual member may include, for example, identifying the individual member based on one or more communications such as the communications (110) in the DCN. In other instances, identifying the individual member may include receiving an identification of the individual member from, for example, a health care professional team. Identifying the individual member may also include identifying the individual member based on a lifestyle driven disease that the individual member may have or have experience with. The lifestyle driven disease may be, for example, a metabolic disease, an auto-immune disease, or a cardiovascular disease.

At Block 206 an intervention is facilitated. The intervention may be the same as or similar to the intervention (114) and may be facilitate for the individual member. Facilitating the intervention may include, for example, providing instructions to the individual member to participate in the intervention when, for example, the intervention is an activity such as the activity (122). In other instances, facilitating the intervention may include providing the intervention when the intervention is an item such as a medical device or a drug. In embodiments where the intervention is a drug, facilitating the intervention may include obtaining a prescription for the intervention.

The intervention may be selected based on an appropriateness for the individual member. The appropriateness of the intervention may be based on, for example, communications in the DCN. In other embodiments, an indication of the appropriateness of the intervention may be received from a health care professional or health care professional team.

At Block 208 instructions to the individual member are provided. The instructions include instructions for the individual member to reflect on using the intervention or lifestyle factors that the intervention enabled the individual member to overcome. The individual member may reflect on the intervention by recording their reflections in one or more communications in the DCN.

At Block 210 a reimbursement is provided to the individual member. The reimbursement may be reimbursement for at least some of a cost of the intervention, the entire cost of the intervention, and/or an out of pocket cost for the intervention. The reimbursement may be provided in response to the individual member recording at least one reflection. In some embodiments, the reimbursement may be provided in response to the individual member recording at least two reflections.

The method described above in FIG. 2 can include more or less steps. The method may also repeat any step or combination or steps.

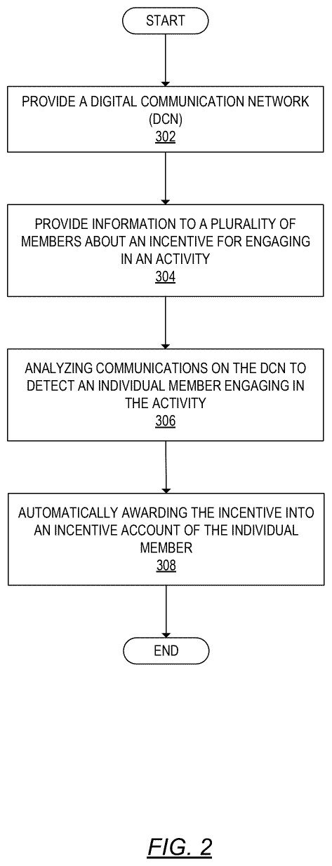

FIG. 3 is a logic flow diagram that illustrates a method, and a result of execution of computer program instructions, in accordance with various embodiments. The method can also be used to incentivize an individual member to reflect on an activity. The method can also, in other instances, be used in part to facilitate or accomplish at least one of: manage the health care cost of the population; reduce the health care risk in the population; and/or slow the progression of an adverse health condition or a lifestyle driven disease. The lifestyle driven disease may be, for example, a metabolic disease, an auto-immune disease, or a cardiovascular disease.

At Block 302, a digital communication network (DCN) is provided. The DCN may be the same as or similar to the DCN (142) and provides a network for members of a population to interact with each other or with the DCN (142).

At Block 304, information is provided to a plurality of members about an incentive for engaging in an activity. The information may be provided via communications such as the communications (110) in the DCN to the plurality of members.

The incentive may be the same as or similar to the incentive (112). The activity may be the same as or similar to the activity (122) and may include a list of activities such as, for example, sharing an experience with a self-selected behavior such as the behavior (124), sharing a reflection on the experience, and/or sharing an outcome of the experience. The behavior may be, for example, a healthy lifestyle behavior, an interaction with a medical system, use of a drug or supplement, use of a medical device, providing a biological sample, and providing a survey or psychological instrument.

At Block 306, communications on the DCN are analyzed to detect an individual member engaging in the activity. The communications may be analyzed by a communications analyzer such as the communications analyzer (136). The communications may include, for example, key words or phrases indicating that the individual member is engaging in the activity.

Detecting that the individual member is engaged in the activity may include detecting that the individual member is participating in a behavior such as, for example, a healthy lifestyle behavior, an interaction with a medical system, use of a drug, supplement, or medical device, providing a survey or psychological instrument, or providing a biological sample. In examples where the behavior is providing the biological sample, the biological sample, may be a saliva sample, a blood sample, and a stool sample. The biological sample may also be provided at the individual member's home and results may be sent to the DCN.

At Block 308, the incentive is automatically awarded into an incentive account of the individual member. The incentive may be awarded in response to detecting that the individual member has engaged in multiple occurrences of the selected activity. Though in other embodiments, the incentive may be awarded in response to detecting that the individual member has engaged in the selected activity once.

The incentive account may be, for example, a bank account or an account on the DCN. The incentive account may be used by the individual member to, for example, acquire an item that is health inducing. The item may be on a list of acquirable items provided to the individual member. The item may be, for example, an intervention such as the intervention (114), health food, a health monitoring device, a biological test, etc.

The method may also include providing instructions to the individual member to reflect on their experience with engaging in the activity and/or their experience with the item. For example, the individual member may include sharing a reflection on use of a health inducing item acquired through earned incentives.

The method also provides a means for an individual member to communicate their health journey with the rest of the healthcare system, particularly their primary care physician. These communications can be automatically generated from the individual member's interactions with the DCN, including posts, experiences, biometric results, discussions and the like. The notes are triggered for production when an individual member indicates (directly or indirectly) that they have an upcoming interaction with the healthcare system. This can include providing questions for the individual member to ask their physician. In this manner, the DCN, through a bot, causes the user to reflect and then assists the individual member during a meeting with the physician by providing the potential questions to ask.

The method described above in FIG. 3 can include more or less steps. The method may also repeat any step or combination or steps.

The methods of FIGS. 2 and 3 can be useful in many scenarios. For example, health care in a population can be improved by providing a means for members of said population to reflect on their health through a DCN. The communications in the DCN can be analyzed to identify an individual member that is participating in an intervention or health-inducing activity and can prompt the individual member to provide their reflection on the intervention or health-inducing activity. The individual member can then be automatically rewarded. Such incentives and reflection provided by the individual member can encourage more members of the population to take part in the intervention or health-inducing activity, thereby improving the overall health of the population.

In another example, the methods can be used to manage a variety of conditions such as, for example, chronic system inflammation (CSI). For example, specific inflammatory markers may be of interest, e.g., in diagnosing and approving drugs to treat rheumatoid arthritis (RA). Such markers may include, for example, C-reactive protein (CRP), which can be combined with other markers. Management of a condition can include maintaining the condition at the present levels, slowing the progression or reversing the condition (if possible). Using the DCN, many members of the population can be assisted which allows the embodiments to be used to manage the health of the population, the health risks of the population and/or reduce health care expense of the population overall.

By way of background, generalized CSI is not specific to any disease and, as such, does not often fit into a single category in a healthcare system. Such characterization results in CSI not being treated in a risk/benefit positive way with drugs or surgery or with any of the tools the provider and insurer community provide. Instead, it is driver and marker of disease progression in general, even if the particular disease is unknown. While the risk-benefit-ratio of drugs and surgery are not positive it can be improved (and therefore disease progression can be slowed) with lifestyle, social situation, emotional management with little or no risk.

Although CSI effects progression of a disease, progression is not typically measured in healthcare. Inflammation and its management impact disease progression and, at some point, disease state. If disease progression can be slowed, the disease state can be avoided and the population is less sick. Thus, it is beneficial to track CSI and whether an intervention has improved or not improved CSI through the individual member's communications and reflections in the DCN.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 4A, the computing system (400) may include one or more computer processor(s) (402), non-persistent storage device(s) (404), persistent storage device(s) (406), a communication interface (408) (e.g., Bluetooth interface, infrared interface, network interface, optical interface, etc.), and numerous other elements and functionalities that implement the features and elements of the disclosure. The computer processor(s) (402) may be an integrated circuit for processing instructions. The computer processor(s) (402) may be one or more cores, or micro-cores, of a processor. The computer processor(s) (402) includes one or more processors. The computer processor(s) (402) may include a central processing unit (CPU), a graphics processing unit (GPU), a tensor processing unit (TPU), combinations thereof, etc.

The input device(s) (410) may include a touchscreen, keyboard, mouse, microphone, touchpad, electronic pen, or any other type of input device. The input device(s) (410) may receive inputs from a user that are responsive to data and messages presented by the output device(s) (412). The inputs may include text input, audio input, video input, etc., which may be processed and transmitted by the computing system (400) in accordance with one or more embodiments. The communication interface (408) may include an integrated circuit for connecting the computing system (400) to a network (not shown) (e.g., a local area network (LAN), a wide area network (WAN) such as the Internet, mobile network, or any other type of network) or to another device, such as another computing device, and combinations thereof.

Further, the output device(s) (412) may include a display device, a printer, external storage, or any other output device. One or more of the output device(s) (412) may be the same or different from the input device(s) (410). The input device(s) (410) and output device(s) (412) may be locally or remotely connected to the computer processor(s) (402). Many different types of computing systems exist, and the aforementioned input device(s) (410) and output device(s) (412) may take other forms. The output device(s) (412) may display data and messages that are transmitted and received by the computing system (400). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (402), is configured to perform one or more embodiments, which may include transmitting, receiving, presenting, and displaying data and messages described in the other figures of the disclosure.

The computing system (400) in FIG. 4A may be connected to, or be a part of, a network. For example, as shown in FIG. 4B, the network (420) may include multiple nodes (e.g., node X (422) and node Y (424), as well as extant intervening nodes between node X (422) and node Y (424)). Each node may correspond to a computing system, such as the computing system shown in FIG. 4A, or a group of nodes combined may correspond to the computing system shown in FIG. 4A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (400) may be located at a remote location and connected to the other elements over a network.

The nodes (e.g., node X (422) and node Y (424)) in the network (420) may be configured to provide services for a client device (426). The services may include receiving requests and transmitting responses to the client device (426). For example, the nodes may be part of a cloud computing system. The client device (426) may be a computing system, such as the computing system shown in FIG. 4A. Further, the client device (426) may include or perform all or a portion of one or more embodiments.

The computing system of FIG. 4A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to manage a health care risk in a population, the method comprising:
providing a digital communication network (DCN) for a plurality of members of a population to communicate with each other;
identifying an individual member of the population with a lifestyle driven disease;
facilitating, through the DCN, an intervention appropriate for the lifestyle driven disease to the individual member;
providing, through the DCN, instructions for the individual member to reflect on at least one of using the intervention or lifestyle factors that the intervention allowed the individual member to overcome; and
providing a reimbursement to the individual member for at least some of a cost of the intervention in response to the individual member recording at least one reflection.

2. The method of claim 1, wherein the intervention comprises at least one of a medical device, a prescription, and a drug.

3. The method of claim 1, wherein identifying the member comprises at least one of identifying the member based on communications by the member in an online social network of the DCN and receiving an identification of the member from a health care professional team.

4. The method of claim 1, wherein the instructions for the member to reflect comprise instructing the member to reflect using one or more communications in the DCN.

5. The method of claim 4, wherein the one or more communications in the DCN comprise at least one of: one-to-one communications, one-to-many communications, one-to-system communications, system-to-one communications, and system-to-many communications.

6. The method of claim 4, wherein the DCN comprises an artificial intelligence bot configured to derive generated communications from at least one of an analysis of existing communications in the DCN or biometrics provided to the DCN.

7. The method of claim 1, wherein the lifestyle driven disease is at least one of: a metabolic disease, an auto-immune disease, and a cardiovascular disease.

8. The method of claim 1, wherein providing the reimbursement to the member comprises reimbursing the member for at least one of: a total cost of the intervention and out-of-pocket cost of the intervention.

9. The method of claim 1, wherein providing the reimbursement to the member is in response to the member recording at least two reflections.

10. A method to manage a health care risk in a population, the method comprising:
providing a digital communication network (DCN) for a plurality of members of a population to communicate with each other;
providing information to the plurality of members of the population about at least one incentive for engaging in a selected activity, wherein the selected activity comprises at least one of: sharing an experience with a self-selected behavior, sharing a reflection on the experience, and sharing an outcome of the experience;
analyzing, by the DCN, communications on the DCN to detect an individual member engaging in the selected activity in the DCN; and
in response to detecting the individual member engaging in the selected activity, automatically awarding, by the DCN, the at least one incentive into an incentive account of the individual member.

11. The method of claim 10, wherein providing information to the plurality of members comprises sending a communication about the at least one incentive through the DCN.

12. The method of claim 10, wherein the DCN comprises at least one of: an online social network, and a conversation bot.

13. The method of claim 10, wherein detecting the individual member engaging in the selected activity comprises detecting the individual member participating in a behavior.

14. The method of claim 13, wherein the behavior is at least one of a healthy lifestyle behavior, an interaction with a medical system, use of a drug or supplement, use of a medical device, providing a biological sample, and providing a survey or psychological instrument.

15. The method of claim 10, wherein automatically awarding the incentive is in response to detecting the individual member engaging in multiple occurrences of the selected activity.

16. The method of claim 10, further comprising enabling the individual member to use the incentives account to acquire an item that is health inducing.

17. The method of claim 16, wherein the item that is health inducing comprises at least one of: health food, a health monitoring device, a biological test, and an intervention.

18. The method of claim 17, further comprising at least one of determining an appropriateness of the intervention for the member based on communications in the DCN and receiving an indication of an appropriateness of the intervention for the member from a health care professional team.

19. The method of claim 17, wherein the intervention is directed to improving at least one of: a metabolic disease, an auto-immune disease, and a cardiovascular disease.

20. A method to manage a health care risk in a population, the method comprising:
providing a digital communication network (DCN) for a plurality of members of a population to communicate with each other;
informing the plurality of members of the population about at least one incentive for engaging in a selected activity, wherein the selected activity comprises participating in a self-selected behavior;
analyzing, by the DCN, communications on the DCN to detect an individual member engaging in the selected activity in the DCN;
receiving a reflection from the individual member regarding the selected activity; and
in response to detecting the individual member engaging in the selected activity and receiving the reflection, automatically awarding, by the DCN, the incentive into an incentive account of the individual member.
